(19) United States
(12) Patent Application Publication
(10) Pub. No.: US 2014/0066665 A1
(43) Pub. Date: Mar. 6, 2014

(54) PROCESS FOR THE PREPARATION OF MENTHOL

(71) Applicant: LANXESS DEUTSCHLAND GMBH, COLOGNE (DE)

(72) Inventors: Martin MECHELHOFF, Koeln (DE); Claus DREISBACH, Leichlingen (DE); Joerg-Dietrich JENTSCH, Leverkusen (DE); Lutz HEUER, Dormagen (DE); Peter WASSERSCHEID, Erlangen (DE); Anne RITTSTEIGER, Erlangen (DE)

(73) Assignee: LANXESS DEUTSCHLAND GMBH, COLOGNE (DE)

(21) Appl. No.: 14/013,434

(22) Filed: Aug. 29, 2013

(30) Foreign Application Priority Data
Aug. 31, 2012 (EP) 12182667.1

(51) Int. Cl. C07C 29/145

(52) U.S. Cl. CPC C07C 29/145
USPC 568/830

(57) ABSTRACT

The invention relates to a process for the preparation of 2-isopropyl-5-methylcyclohexanol (D,L-menthol) via the hydrogenation of thymol to menthone and subsequent further hydrogenation to give D,L-menthol.

The invention relates to a process for the preparation of 2-isopropyl-5-methylcyclohexanol (menthol) via the hydrogenation of thymol to menthone and subsequent further hydrogenation to give menthol (D,L-menthol).

2-Isopropyl-5-methylcyclohexanol has three stereogenic centres, therefore giving eight stereoisomers: D,L-menthol, D,L-neomenthol, D,L-isomenthol and D,L-neoisomenthol.

Among the naturally occurring cyclic terpene alcohols, L-menthol, the main constituent of peppermint oil, assumes a special position on account of its cooling and refreshing effect. L-menthol is therefore used as a fragrance or flavouring and is used in the pharmaceutical industry. It is therefore the most economically important of the menthol stereoisomers. The general aim has therefore been to carry out the hydrogenation through suitable selection of the reaction conditions and the catalysts such that as much D,L-menthol as possible is formed.

Many substance mixtures whose components have only slight differences in boiling point or even form azeotropes can only be separated with difficulty, if at all, by conventional rectification. This applies to the separation of diastereomers of 2-isopropyl-5-methylcyclohexanol from substance mixtures comprising at least two diastereometric compounds of 2-isopropyl-5-methylcyclohexanol relative to one another, as are typically formed during the hydrogenation of thymol or subsequent work-up steps. In particular, the separation of the diastereomers isomenthol and menthol can only be performed inadequately and with a high input of energy on account of the low relative volatility of the two compounds relative to one another.

The boiling points of D,L-isomenthol (218.6° C. at 1013 hPa; 75 to 78° C. at 3.3 hPa) and D,L-menthol (216.5° C. at 1013 hPa; 75 to 78° C. at 3.3 hPa) are very close to one another. The separation efficiency of a column during the distillative separation of the individual menthol isomers is therefore determined in particular by the ratio of D,L-menthol to D,L-isomenthol. For a high space-time yield of D,L-menthol during the distillative separation, besides an extremely high D,L-menthol content in the mixture to be separated, an extremely low D,L-isomenthol content is therefore also required. The yield of menthol is thus determined for a given distillation column essentially by the starting ratio of D,L-menthol to D,L-isomenthol.

To produce D,L-menthol, it is known to hydrogenate compounds having the carbon skeleton of menthane with at least one double bond and having oxygen substitution in the 3 position, such as, for example, thymol, in continuous processes over fixed catalyst beds with hydrogen and/or to rearrange stereoisomers of menthol over fixed catalyst beds.

DE 23 14 813 A 1 describes a process for hydrogenating compounds having the carbon skeleton of menthane with at least one double bond and having oxygen substitution in the 3 position over a bed of a cobalt-manganese catalyst at temperatures of 170° C. to 220° C. and a pressure exceeding 25 bar, preferably exceeding 200 bar. In the examples, temperatures of 180° C. to 210° C. and pressures above 200 bar are employed, and a mixture of the eight stereoisomeric menthols is obtained which consists to 59.5 to 59.9% of the racemic D,L-menthol and to 10.6 to 10.8% of D,L-isomenthol. The maximum menthol/isomenthol ratio is 5.7. By modifying the cobalt-manganese catalyst with copper, menthol mixtures with D,L-menthol contents of 57.6% and D,L-isomenthol contents of 9.2% were achieved, which corresponds to a menthol/isomenthol ratio of about 6.3. The resulting mixtures, however, have 4 to 5% of undesirable by-products in the form of non-reutilizable hydrocarbons.

EP 0 563 611 A 1 and DE 197 18 116 A 1 disclose that the hydrogenation of aromatic or partly hydrogenated cyclic compounds having the carbon skeleton of menthane with at least one C═C double bond and having oxygen substitution in the 3 position can be performed with hydrogen over a fixed bed catalyst comprising palladium, ruthenium or rhodium or a mixture of these elements as active constituents and alkali metal hydroxides and/or sulphates as promoters, in each case applied to a support, the support being doped with a metal from the rare earths and manganese. In the examples, temperatures of 180 to 240° C. and pressures of 270 to 300 bar were employed. Here, menthol mixtures were obtained which forms approx. 52 to 57% D,L-menthol and 11.5 to 14.8% D,L-isomenthol, which corresponds to a menthol/isomenthol ratio of 3.6 to 4.4.

EP 743 296 A 1 discloses catalysts which consist of support-free, compressed powders of cobalt oxides or hydroxides, manganese oxides or hydroxides and alkaline earth metal oxides or hydroxides, and are used at temperatures of 150° C. to 230° C. and pressures of 25 to 350 bar.

The rearrangement of stereoisomers of 1-menthol is described in U.S. Pat. No. 5,756,864: At temperatures of from 200 to 350° C. and hydrogen pressures of 50 to 350 bar, preferably 100 to 300 bar, D-menthol is racemized and isomerized in a continuous process over a catalyst, where the catalyst consists of support-free, compressed powders of nickel hydroxides or oxides, manganese hydroxides or oxides and alkaline earth metal hydroxides or oxides. Here, menthol mixtures were obtained which consisted to a maximum of 59.8% of D,L-menthol.

U.S. Pat. No. 2,843,636 discloses carrying out the isomerization of stereoisomers of menthol to give D,L-menthol with hydrogen in the presence of a hydrogenation catalyst from the group copper chromite, cobalt and nickel at 260 to 280° C. and 500 to 1300 p.s.i.g. (34 to 90 bar) in autoclaves. As well as approx. 10 to 12% D,L-isomenthol, the resulting mixtures have a D,L-menthol content of 60 to 64%.

DE 198 53 562 A describes a low-pressure hydrogenation of thymol over a stationary catalyst bed having a temperature gradient: The first two of five serially connected tubular reactors are heated to 180° C., the three tubular reactors behind being heated to 80 to 90° C. Using a catalyst which contains, on a support doped with a metal from the rare earths and with manganese, ruthenium as active constituent and alkali metal hydroxides as promoters, it was possible, at a pressure of 3 bar, to obtain a menthol isomer mixture which comprised 64.4% by weight menthol and 12.1% isomenthol, which corresponds to a menthol/isomenthol ratio of 5.3. Isomerization of a hydrogen-saturated mixture of D,L-neomenthol, D,L-isomenthol and D,L-menthol produced, at atmospheric pressure, an isomer mixture with a composition of 65.3% D,L-menthol and 12.1% isomenthol. In this low-pressure process, it is possible to achieve high menthol contents of approx. 65%. The maximum menthol/isomenthol ratio, however, is 5.4.

DE 100 23 283 A now discloses an improved process in which isomer mixtures which typically have about 55% D,L-menthol can prepare, by means of isomerization with simple supported ruthenium catalysts, menthol-richer mixtures which have up to 67.3% D,L-menthol and only 8.2% D,L-isomenthol, i.e. a menthol/isomenthol ratio of up to 8.1. Furthermore, DE 100 23 283 A discloses that the catalysts can be regenerated with alcoholates, oxides and hydroxides of the alkali metals or alkaline earth metals.

Accordingly, a common aspect of all of the known processes is that they only permit a maximum fraction of around 60% of D,L-menthol, produce at least 8.2% D,L-isomenthol and permit maximum menthol/isomenthol ratios of 8.1.

It was therefore an object of the invention to provide a selective and technically simple process for the preparation of D,L-menthol in high yields, in which ideally no or only small amounts of D,L-isomenthol are formed and which permits high menthol/isomenthol ratios, with the formation of undesired by-products largely being avoided at the same time.

Surprisingly, the object has been able to be achieved by a 2-stage hydrogenation in which, to in the first selective hydrogenation, thymol is converted to the ketones iso-/menthone.

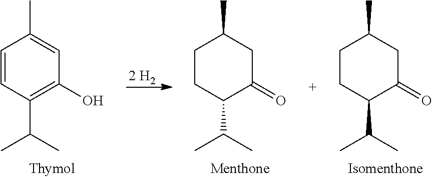

and, after distillative separation of the two ketones, the resulting menthone is then hydrogenated again.

The present invention provides a process for the preparation of 2-isopropyl-5-methylcyclohexanol (menthol), characterized in that
a) thymol is hydrogenated with hydrogen in the presence of a catalyst selected from the group of the elements of group VIII b (iron-platinum group) of the Periodic Table of the Elements, preferably Pt, Rh, Ru, Pd, particularly preferably Pd,
   optionally in the presence of a solvent,
b) the 2-isopropyl-5-methylcyclohexanone (menthone) isolated from a) is hydrogenated with hydrogen in the presence of a catalyst selected from group VIII b (iron-platinum group), preferably Pt, Rh, Ru, Pd, particularly preferably Pd, as supported or unsupported catalysts, optionally in the presence of a solvent to give menthol,
c) the neomenthol which is formed alongside menthol is separated off and
e) optionally then an isomerization of neomenthol to menthol is carried out.

In a preferred embodiment of the process according to the invention, the stages a) and/or b) are carried out at temperatures of from 60°-200° C., particularly preferably 60-120° C. and at a pressure of at least 1.1 bar, preferably >1.1 to 325 bar, particularly preferably 2-100 bar, very particularly preferably 10 to 30 bar.

In a preferred embodiment of the present invention, in step a), a 2 to 150-fold molar excess of hydrogen is used per 1 mol of thymol.

In a preferred embodiment of the process according to the invention, the catalysts for the preparation of menthone in step a) are used as supported or unsupported catalysts, particularly preferably as supported catalysts.

Preferred support materials are metal oxides and activated carbon. Particular preference is given to SiO₂, AlO₃, TiO₂, ZrO₂ or sulphates, and therein preferably BaSO₄, or mixtures thereof and activated carbon. Very particular preference is given to Al₂O₃ and activated carbon, BaSO₄, Al₂O₃ and/or silica. In a further particularly preferred embodiment of the invention, the support material is made of Al₂O₃ and silica and/or activated carbon.

The support material preferably has a BET surface area of at least 100 m²/g, preferably at least 160 m²/g, particularly preferably at least 180 m²/g. Particular preference is given to aluminium oxide which additionally has a high fraction of macroporous pores with a pore diameter of at least 50 nm and has a pore volume of at least 300 mm³/g, preferably at least 600 mm³/g.

The fraction of the catalyst based on the support material is preferably 0.3-10% by weight, particularly preferably 2-5% by weight.

Very particular preference is given to a support material based on Al₂O₃ and silica with a fraction of 2-5% by weight palladium.

The catalysts are standard commercial catalysts which are obtainable e.g. from Heraeus Materials Technology GmbH & Co. KG or Johnson Matthey Plc.

In a further preferred embodiment of the invention, step a) is carried out in a solvent.

Preferred solvents are cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether.

Particular preference is give to cyclohexane.

The ratio of thymol to solvent is preferably 1:0 to 1:20.

In a further embodiment of the invention, the catalyst used in step a) can be recycled. For this purpose, preference is given to using continuous through-flow reactors, preferably fluidized-bed reactors or reactors with a fixed catalyst bed.

The menthone which is formed in step a) is preferably separated off by distillation at temperatures of from 50 to 150° C. The distillation bottom, comprising isomenthone and small fractions of by-product, is preferably returned and converted via a keto-enol tautomerism into the thermodynamic equilibrium of iso-/menthone, and separated again by distillation into menthone and isomenthone.

Suitable catalysts for establishing the keto-enol tautomerism are preferably the oxides and/or hydroxides of the elements: aluminium, magnesium, iron, zinc and silicon. Particular preference here is given to basic aluminium oxide and magnesium oxide.

The catalysts used for establishing the keto-enol tautomerism are standard commercial catalysts which are available e.g. from Merck KG or from Lanxess Deutschland GmbH.

The keto-enol tautomerism into the thermodynamic equilibrium of iso-/menthone is preferably carried out at temperatures of from 0 to 100° C., particularly preferably at 20 to 75° C.

In a further preferred embodiment of the invention, this rearrangement (the keto-enol tautomerism) takes place in a solvent.

Preferred solvents are cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether.

Particular preference is given to cyclohexane.

The ratio of thymol to solvent is preferably 1:0 to 1:20.

In a further hydrogenation step (step b)), the menthone isolated from a) is hydrogenated with hydrogen in the presence of a catalyst selected from group Viii b (iron-platinum group) as supported or unsupported catalysts, preferably Pt, Rh, Ru, Pd, particularly preferably Rh, optionally in the presence of a solvent, to give menthol, which is formed in a mixture with neomenthol:

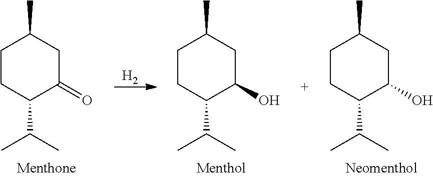

The unreacted neomenthol can then be returned and converted in an epimerization/isomerization to menthol.

In a preferred embodiment of the present invention, in step b), a 2 to 150-fold molar excess of hydrogen is used per 1 mol of menthone.

In a preferred embodiment of the process according to the invention, the catalysts for the preparation of menthone (step b)) are used as supported or unsupported catalysts, particularly preferably as supported catalysts.

Preferred support materials are metal oxides and activated carbon. Particular preference is given to SiO₂, Al₂O₃, TiO₂, ZrO₂ and sulphates, and therein preferably BaSO₄, or mixtures thereof and activated carbon. Very particular preference is given to Al₂O₃, activated carbon, BaSO₄ and/or silica. In a further particularly preferred embodiment of the invention, the support material is particularly preferably made of Al₂O₃ and silica and/or activated carbon.

The support material preferably has a BET surface area of at least 100 m²/g, preferably at least 160 m²/g, particularly preferably at least 180 m²/g. Particular preference is given to aluminium oxide which additionally has a high fraction of macroporous pores with a pore diameter of at least 50 nm and has a pore volume of at least 300 mm³/g, preferably at least 600 mm³/g.

The fraction of the catalyst based on the support material is preferably 0.3-10% by weight, particularly preferably 2-5% by weight.

Very particular preference is given to a support material made of Al₂O₃ with a fraction of 2-5% by weight ruthenium.

The catalysts are standard commercial catalysts which are available e.g. from Alfa Aesar GmbH.

During the hydrogenation, the temperatures are preferably from 60°-200° C., particularly preferably 60-120° C.

During the hydrogenation, the pressure is preferably at least 1.1 bar to 325 bar, particularly preferably 2 to 100 bar.

In a further preferred embodiment of the invention, step c) is carried out in a solvent.

Preferred solvents are cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether.

Particular preference is given to cyclohexane.

The ratio of menthone to solvent is preferably 1:0 to 1:20.

During the hydrogenation, neomenthol is also formed alongside menthol.

In step c), the neomenthol is separated off from this menthol mixture. This separation is preferably carried out by distillation at temperatures of 60 to 150° C.

In a preferred embodiment of the present invention, the separated-off neomenthol is converted to menthol in a subsequent step in an isomerization reaction.

For this purpose, preference is given to using the isomerization catalysts based on ruthenium and alkaline earth metal alkoxylate described in WO2012/010695, which are applied to a support material made of aluminium oxide.

Preferred alkaline earth metal alkoxylates are compounds of the formula (I),

(R—O)₂M (I),

in which
R is in each case independent but preferably identical, and is a primary, secondary or tertiary, cyclic or acyclic, branched or unbranched C₁ to C₂₀-alkyl radical which can optionally be further substituted by aryl, C₁-C₄-alkoxyl or C₆ to C₁₄-aryloxy and is particularly preferably methyl, ethyl, n-propyl, isopropyl, n-butyl, sec-butyl, tert-butyl, n-pentyl, neopentyl, n-hexyl, cyclohexyl or the stereoisomeric methyl radicals and
M is calcium, strontium or barium, preferably barium.

The preferred barium alkoxylates can be obtained for example by reacting barium perchlorate with the corresponding potassium alkoxylates, preferably dissolved in the same alcohol or a different alcohol, whereupon sparingly soluble potassium perchlorate is formed which can be removed easily from the reaction solutions for example by filtration.

Barium mentholates are for example also obtainable by admixing barium ethoxide or barium isopropoxide with an excess of menthol stereoisomers and prolonged standing or heating.

Particular preference is given to using barium ethoxide, 10% w/v in ethanol, barium isopropoxide as a solid substance, dissolved in menthol isomers, or barium isopropoxide, 20% w/v in isopropanol.

The aluminium oxide used as the support material can be used in all known modifications, preferably in the γ modification. The aluminium oxide used as support material advantageously has a BET surface area of at least 100 m²/g, preferably at least 160 m²/g, particularly preferably at least 180 m²/g. Particular preference is given to aluminium oxide, which additionally has a high fraction of macroporous pores with a pore diameter of at least 50 nm and has a pore volume of at least 300 mm³/g, preferably at least 600 mm³/g. Examples of suitable support materials include the commercially available aluminium oxides SPH 1515, SPH 531, SPH 501 from Rhodia, D 10-10 from BASF and SA 6176 from Norton.

The support material can be used for example in the form of powders with particle sizes of from 0.001 to 0.1 mm, crushed and sieved material with particle sizes between 0.05 and 5 mm or in mouldings, preferably extrudates, pellets, beads or granules with diameters of from 0.2 to 30 mm.

The particular advantage of the process according to the invention is that mixtures of diastereomers of 2-isopropyl-5-methylcyclohexanoles can be separated in an efficient manner such that menthol from the diastereomers menthol and menthol is obtained with high purity.

In the process according to the invention, the specific energy consumption can be considerably lowered and the dimensions of the separation apparatuses used, i.e. the required apparatus volume per required separation stage, can be considerably reduced.

The scope of the invention encompases all of the above and below general or preferred radical definitions, indices, parameters and explanations with one another, i.e. also between the respective ranges and preferred ranges in any desired combination.

The examples which follow serve to illustrate the invention but have no limiting effect.

### EXAMPLES

### Hydrogenation to Give Menthone

### Example 1

6 g of thymol (40 mmol) were hydrogenated with an at least 2 molar excess of hydrogen in the presence of 3 mol % of Cat L1 to L3 (see table below) at temperatures of 120° C. and at a pressure of 10 bar in the presence of cyclohexane as solvent to give 2-isopropyl-5-methylcyclohexanone (menthone).

| Catalyst | Manufacturer | Name |
|---|---|---|
| 5% Pd/Alox | Heraeus | L1 |
| 4% Pd/Alox-Silica | Heraeus | L2 |
| 2% Pd/Alox-Silica | Heraeus | L3 |

where Heraeus = Heraeus Materials Technology GmbH & Co. KG, Alox = aluminium oxide

The menthone was separated off by distillation at a bottom temperature of 133° C.

Under the stated conditions, catalysts L1 and L2 exhibited a very similar reaction rate. Complete conversion was achieved after about 30 min. By contrast, the activity of catalyst L3 was considerably higher. Here, complete conversion was achieved after just 5 minutes.

Catalysts L1 and L2 exhibited a ketone selectivity of more than 97% over almost the entire conversion range. At complete conversion, L3 even still produced values around 99%.

Catalysts L1 and L2 also exhibited very similar behaviour as regards the menthone selectivity. Here, the menthone selectivity increased over the entire conversion range continuously up to a maximum value of about 68%. In the case of catalyst L3, the menthone selectivity only increased significantly at complete conversion.

In the case of catalysts L1 and L2, the fraction of isomenthol increased to about 0.12%, in the case of catalyst L3 a value of 0% was still able to be achieved even shortly before reaching complete conversion.

Hydrogenation to give menthone:

### Example 2

6 g of thymol (40 mmol) were hydrogenated with an at least 2 molar excess of hydrogen in the presence of 2.5 mol % of Cat L3 to L9 (see table below) at temperatures of 120° C. and at a pressure of 10 bar in the presence of cyclohexane as solvent to give 2-isopropyl-5-methylcyclohexanone (menthone).

| Catalyst | Manufacturer | Name |
|---|---|---|
| 5% Pd/Alox Type 325 | Johnson Matthey Plc | L4 |
| 5% Pd/Alox | Johnson Matthey Plc | L5 |
| 5% Pd/Alox | Johnson Matthey Plc | L6 |
| 5% Pd/Alox | Johnson Matthey Plc | L7 |
| 5% Pd/Alox | Johnson Matthey Plc | L8 |
| 5% Pd/BaSO₄ | Johnson Matthey Plc | L9 |
| Type A201053 | | |
| 2% Pd/Alox-Silica | Heraeus | L3 |

where Heraeus = Heraeus MaterialsTechnology GmbH & Co. KG

Very good selectivities between 96 and 98% were consistently achieved with all of the tested catalysts. The best results were achieved with catalysts L3, L5 and L9.

Very low isomenthol fractions of less than 0.17% were consistently achieved with all of the tested catalysts. The best results were shown by catalysts L3, L6 and L9 with isomenthol fractions of about 0.1% at complete conversion. In the case of catalyst L3, still no isomenthol at all could be detected even just before reaching complete conversion.

### Example 3

### Hydrogenation of Menthone to Menthol

The menthone formed in Example 1 sample L3 was separated off by distillation at a bottom temperature of 133° C. and hydrogenated to menthol at 120° C. and 30 bar. The catalyst used was 5% Ru/Alox reduced from Alfa Aesar. The reaction was carried out in the solvent cyclohexane.

Menthone had reacted completely after a good 3 h. Neomenthol and menthol were formed in approximately equal fraction. The fraction of the undesired products iso- and neoisomenthol is negligibly small.

What is claimed is:

1. Process for the preparation of 2-isopropyl-5-methylcyclohexanol (D,L-menthol), characterized in that
a) thymol is hydrogenated with hydrogen in the presence of a catalyst selected from the group of the elements of group VIII b (iron-platinum group), preferably Pt, Rh, Ru, Pd, particularly preferably Pd, optionally in the presence of a solvent,
b) the 2-propyl-(2)-5-methylcyclohexanone (menthone) isolated from a) is hydrogenated with hydrogen in the presence of a catalyst selected from group VIE b (iron-platinum group), preferably Pt, Rh, Ru, Pd, particularly preferably Rh, as supported or unsupported catalysts, optionally in the presence of a solvent to give menthol, and
c) the neomenthol which is formed alongside menthol is separated off and
d) optionally then an isomerization reaction of neomenthol to give menthol is carried out, where stages a) and/or c) are carried out at temperatures of from 60° to 200° C. and at a pressure of at least 1.1 bar.

2. Process according to claim 1, characterized in that the catalysts used for the preparation of menthone are supported or unsupported catalysts.

3. Process according to claim 2, characterized in that the supports used for the supported catalysts are metal oxides and activated carbon, preferably SiO₂, Al₂O₃, TiO₂, ZrO₂ or sulphates, preferably BaSO₄, or mixtures thereof and activated carbon, particularly preferably Al₂O₃ and activated carbon.

4. Process according to one or more of claims 1 to 3, characterized in that the solvents used are cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether.

5. Process according to one or more of claims 1 to 4, characterized in that the isomerization takes place with ruthenium and alkaline earth metal alkoxylate which have been applied to a support material made of aluminium oxide.
